(19) United States
(12) Patent Application Publication
(10) Pub. No.: US 2024/0139335 A1
(43) Pub. Date: May 2, 2024

(54) EXINE CONSTRUCTS

(71) Applicant: BOTANICAL SOLUTIONS LIMITED, Bridlington East Riding of Yorkshire (GB)

(72) Inventors: Grahame MacKenzie, Bridlington East Riding of Yorkshire (GB); Terence Simon Corbitt, Bridlington East Riding of Yorkshire (GB); Guy Monroe Entract, Thessaloniki (GR)

(73) Assignee: BOTANICAL SOLUTIONS LIMITED, Bridlington East Riding of Yorkshire (GB)

(21) Appl. No.: 18/278,897

(22) PCT Filed: Feb. 25, 2022
(86) PCT No.: PCT/GB2022/050518

§ 371 Date: Aug. 25, 2023

(30) Foreign Application Priority Data
Feb. 26, 2021 (GB) 2102748.7

(51) Int. Cl. A61K 47/69; A61K 8/97; A61K 47/51; A61Q 19/00

(52) U.S. Cl. CPC A61K 47/6925; A61K 8/97; A61K 47/51; A61Q 19/00

(57) ABSTRACT

An exine-construct that can be coloured and used as a protection and/or delivery and/or removal vehicle for an active substance, or as an antioxidant. It can be used in a method of surgery, therapy, prevention or diagnosis. The invention provides an exine-construct comprising of exine shells together with an active substance; and a method for preparing the exine-construct by isolating exine shells from a naturally occurring spore and suitably attaching them together.

### FIELD OF THE INVENTION

This invention relates to methods for suitably binding exine shells together involving chemical attachment and/or physical attachment to the exines to provide an ‘exine-construct’ that can be coloured and usable as a protection and/or delivery and/or removal vehicles for active substances. Chemical attachment may encompass covalent or other forms of chemical bond, for example sulfide linkages, ionic bonds or dative bonds) or physical attachment (for example hydrogen bonds, van der Waals bonds or hydrophobic and/or hydrophilic interactions). An ‘exine-construct’ comprises exine shells extracted from pollen grains and/or spores combined or linked chemically and/or or physically to each other (directly or by bridging functional groups), in any arrangement or combination, as building blocks, to form a structure of any shape or size.

### BACKGROUND TO THE INVENTION

The size limitation of many plant-based microcapsules makes the storage and/or delivery of a larger amount of substance or of larger sized substances difficult. Such substances need to be formulated in a vehicle, which is large enough but suitable both for their storage, protection and/or subsequent delivery.

It is known from WO-2005/000280 to use the exine coatings of naturally derived (typically plant) spores or pollen grains as delivery vehicles for active substances such as pharmaceuticals and dietetic substances. These coatings can be isolated from spores or pollen grains by successive treatments, for example with organic solvents, alkali and acid so as to remove the lipid, carbohydrate, protein and nucleic acid components that may be attached to or contained within the exine shell. Enzymatic methods have also been used to isolate the exine coating from other components of a spore or pollen grain.

Exine coatings (or shells) take the form of essentially hollow capsules that can be impregnated or filled with, or chemically or physically bound to, another substance. According to WO-2005/000280, a pharmaceutical or dietetic active substance may be physically or chemically bound to, adsorbed on or more typically encapsulated within such a hollow exine shell. The exine/active substance combination may then be formulated—often mixed with conventional excipients, diluents or carriers and/or with release rate modifiers—for the desired mode of delivery; for example, oral, buccal or pulmonary delivery.

Sometimes, when formulating an active substance, it is necessary to protect the substance, at least temporarily, from external influences such as heat, light, moisture or oxygen (air). This may be for the purpose of improving the storage stability of the formulation, or it may be to ensure that the formulation reaches, following its delivery to a plant or human or animal patient, the appropriate part of the plant or body.

Exine shells can themselves provide a degree of protection for an encapsulated active substance; for instance, from atmospheric effects such as light and/or oxygen (air), and therefore from premature degradation. The physical protection they provide can also help reduce loss of the active substance by evaporation, diffusion or leaching. It has also been found (as disclosed in WO-2007/012856) that in many cases an exine shell can itself act as an antioxidant, rather than merely as a physical barrier to sunlight or oxygen (air); this effect being observable even when an active substance is outside of, rather than encapsulated within, the shell.

Useful exine shell coatings may be isolated from spores or pollen grains by treatment with organic solvents, alkali and acid to remove the lipid, carbohydrate, protein and nucleic acid components that may be attached to or contained within the exine shell.

In WO-2007/012856 such exine shells isolated by acid hydrolysis (for example, with phosphoric acid) followed by base hydrolysis (for example, with potassium hydroxide) are designated “AHS”. Also disclosed are exine shells designated “BHS”, which were subjected only to base hydrolysis (for example, with aqueous potassium hydroxide). The BHS samples comprised not only the exine shell but also a proportion of the cellulosic intine layer normally removed during the acid treatment. It was found that some BHS exine coatings were particularly effective in reducing the oxidation rates of oils encapsulated therein when exposed to UV light and air.

In WO 2010/004334 A2, whitened exine shells were obtained in multi-pot processes using, for example a base to form a BHS exine (using for example, aqueous sodium hydroxide or potassium hydroxide) and/or an acid to form an AHS exine (using for example, glacial acetic acid, hydrochloric acid, sulfuric acid, phosphoric acid or hydrochloric acid). The subsequent BHS or AHS was then treated with an oxidant such as sodium chlorate or sodium hypochlorite.

The present inventors have found surprisingly that exine shells prepared by a variety of methods may suitably be bound together to provide an exine-construct that can protect, store and/or deliver active substances.

### Statements of the Invention

According to a first aspect of the present invention there is provided an exine-construct comprising a plurality of exine shells bound together.

According to this aspect of the invention the exine shells may be chemically and/or physically bound together, or a combination thereof, e.g. to form an exine-construct structure. The exine-construct may be formed into a structure wherein said exine-construct structure comprises a well or one or more inner chambers. In one aspect of the invention the exine shells may be chemically bound together. In another aspect of the invention the exine shells may be physically bound together.

According to second aspect of the present invention there is provided a method for suitably binding exine shells together involving chemical attachment (meaning: covalent or other forms of chemical bond, for example sulfide linkages, ionic bonds, or dative bonds) and/or physical attachment (for example hydrogen bonds, van der Waals bonds or hydrophobic and/or hydrophilic interactions) of the exines either directly or via bridging (or spacer) functional groups to provide an exine-construct. The exine-construct structure may for example, take the form of a flat sheet, disc, rod or plate, tablet, capsule, box, lozenge or open bowl (well) and may have one or more hollow chambers, spheres, bricks, sinters, frits and filters. The method of the invention may comprise binding exine shells together involving attachment of the exines either directly or via bridging (or spacer) functional groups to provide an exine-construct, e.g., chemically and/or physically. The method may also comprise compressing the exine shells to provide an exine-construct.

Reference herein to an active substance associated, e.g. loaded in or physically or chemically attached, to an exine-construct should be construed as meaning the active substance being associated with an exine-construct, an exine-construct structure or a combination thereof.

An active substance may be (i) attached physically or chemically to the external surface of the exine shells; (ii) encapsulated within the exine shells; (iii) attached physically or chemically to the external surface of the exine-construct; (iv) loaded and/or encapsulated within the exine-construct chamber, or (v) chemically or physically bound to the inner chamber surface of the exine-construct. An exine-construct may be any of the encapsulated forms (i) to (v) combined or linked chemically to each other (directly or by bridging functional groups), in any arrangement or combination, as building blocks, to form a structure of any shape or size.

An exine-construct may also be the chemical attachment of exines already derivatised (e.g. including but not limited to esters, amides, amines, azides, carboxylates, acids, protonation, sulfonates, sulfides, sulfates, sulfation, thiolation, alkylation, azidation, phosphates, nitrates, metal chelates and halogenates) or where the derivatised exines are encapsulated with an active (e.g. pharmaceutical or cosmetic product) or filler (e.g. magnetite or a polymer such as a cyanoacrylate). The derivatised exine-construct may comprise a bioconjugate, that is, a macromolecular complex obtained synthetically by chemically bonding, e.g. covalently bonding drug molecules to exines within or on the surface of the exine-construct. Although covalent bonding or ionic bonding is preferred for some applications physical attachment such as hydrogen bonding, van der Waals forces and hydrophobic and/or hydrophilic interactions can be included. Added protection of the physically attached or chemically attached exines surfaces of the exine-constructs may be achieved by an additional coating such as with gum Arabic or starch. Conventional film coatings may be used, for example, hydroxypropyl cellulose or other modified celluloses.

In an exine-construct of the invention the exine shells may be derivatised. The exine shells may be derivatised by hydrolysis, salt formation, protonation, deuteration, tritiation, esterification, amination, quarternisation, acetylation, sulfonation, sulfation, thiolation, alkylation, azidation, phosphorylation, nitration, metal chelation, halogenation, hydrogenation or chloromethylation or thiolation, or any combination thereof.

The active substance may be either base or acid labile.

A third aspect of the invention provides an exine-construct according to the first aspect, for use in a method of surgery, therapy, prevention or diagnosis practised on a living human or animal body. The exine-construct may thus be used as a protection and/or delivery vehicle for an active substance and/or the products or secretions of an active substance, which is active as a pharmaceutical or diagnostic agent. One, non-limiting, example of an active substance and/or the products or secretions of an active substance, shall include a hormone producing cell or a living cell that can differentiate into a hormone producing cell, the hormone produced by a hormone producing cell or a combination thereof.

A fourth aspect of the invention provides the use of an exine-construct as herein described as a protection and/or delivery vehicle for an active substance. According to this aspect of the invention there is also provided the use of an exine-construct in the manufacture of a medicament. There is also provided the use of an exine-construct according to the first aspect, in the manufacture of a medicament for the delivery of a pharmaceutically active substance or a diagnostic agent to a human or animal patient.

There is further provided the use of an exine-construct as herein described in the manufacture of a formulation for the protection and/or delivery of an active substance to a plant, human or animal.

There is also provided the use of an exine-construct as herein described as a protection and/or delivery vehicle for a living or dead human, animal, algal, bacterial, fungal, protozoan, or plant cell or virus.

According to fifth aspect of the present invention there is provided a method for protecting an active substance from oxidation and/or for increasing the stability of the active substance or of a construct containing it, the method comprising formulating the active substance with an exine-construct according to the first aspect of the invention. The method may comprise adding an exine-construct to the substance; for example to increase the oxidative stability of a substance.

According to a sixth, aspect of the present invention the exine-construct, or a fragment thereof, can be used:
(i) to load an active (e.g., an active oil from an oil-in-water emulsion);
(ii) in the purification of substances from such as fermentation broths or for the remediation of metals;
(iii) in the removal of substances from solutions and emulsions;
(iv) in the removal or remediation of pollutants and contaminants (e g, oils and pesticides).

The exine-construct may be broken or damaged in parts; the invention thus embraces a fragment of an exine-construct. Such broken or damaged fragments of an exine-construct may also be useful in aspects of the present invention.

The term “removal” shall include, but shall not be limited to, remediation, sequestration, and the like.

According to a seventh, aspect of the present invention the exine-construct, or a fragment thereof can be used for components of machines and electrical/heat insulation units normally fabricated out of synthetic polymers.

An exine shell is the outer coating from around a naturally occurring (“raw”) spore or pollen grain. It may consist in part or mainly of sporopollenin. It may be of a type described in WO-2005/000280, in particular at pages 4, 8 and 9 and in Example 1.

According to the present invention, the exine shell may be derived from any suitable naturally occurring spore or pollen grain, or mixture thereof. In this context, the term “plant” is to be construed in its broadest sense, and embraces for example, algae, angiosperms, fungi, gymnosperms, mosses and pteridosperms. Moreover, the term “spore” is used to encompass not only true spores such as are produced by ferns, mosses and fungi, but also pollen grains, as are produced by seed-bearing plants (spermatophytes) and also endospores of organisms such as bacteria. Similarly, the term “naturally occurring” means that a spore is produced by a living organism, whether prokaryote or eukaryote and whether plant or animal. The spore (which term includes pollen grains and endospores of organisms such as bacteria) may for instance be derived from a plant, or from a fungus, alga or bacterium or another microorganism.

Suitable organisms from which such spores may be obtained include the following, with the approximate diameters of their spores, as published in the literature. Figures in brackets indicate the diameters measured by the inventors, where these differ from published values.

| | | |
|---|---|---|
| Bacillus subtilis | 1.2 | μm |
| Myosotis (“forget-me-not”) | 2.4 (5) | μm |
| Aspergillus niger | 4 | μm |
| Penicillium | 3 (5) | μm |
| Cantharellus minor | 4 (6) | μm |
| Saccharomyces cerevisiae | 6 | μm |
| Ganomerma | 5 (6.5) | μm |
| Agrocybe | 10 (14) | μm |
| Urtica dioica | 10 (12) | μm |
| Periconia | 16 (18) | μm |
| Epicoccum | 20 | μm |
| Ryegrass “Lolium perenne” | 21 | μm |
| Timothy grass | 22 | μm |
| Rye | 22 | μm |
| Lycopodium clavatum | 34 | μm |
| “Lycopodium powder” | 40 | μm |
| Maize “Zea mays” | 80 | μm |
| Hemp “Cannabis sativa” | 24 | μm |
| Rape hemp | 25 | μm |
| Wheat | 23 | μm |
| Abies | 125 | μm |
| Cucurbitapapo | 200 | μm |
| Cuburbita | 250 | μm |

Of these, Lycopodium clavatum, lycopodium powder, pine, ryegrass, rye, sunflower, Timothy grass, Ambrosia trifida, Ambrosia artemisiifolia, hemp, rape, wheat and maize spores may be preferred. Other spores from which exine shells may be extracted are disclosed in the publications referred to at page 8 of WO-2005/000280.

The exine shell may have a diameter (which may be determined by scanning electron microscopy or laser particle size analysis) of about 1 μm or greater, or of about 3 or 5 or 8 or 10 or 12 or about 15 μm or greater. It may have a diameter of up to about 300 μm, or of up to about 250 or 200 or 150 or 100 or 80 or 50 or about 40 μm. For example, its diameter may be from 1 to 300 μm, or from 1 to 250 μm, or from 3 to 80 μm, or from 3 to 50 jam, or from 15 to 40 μm. Grass pollen-derived exines, as well as other exine shells of approximately 20 μm diameter, might also be expected to be suitable, as may pollen exines having diameters of up to around 80 μm.

In applications for which proteins represent potentially allergenic components of the original spore, it may be preferred that the exine shells comprising the exine-construct have a % N level by weight of about 10% or less, or of about 8 or 6 or 4 or 2, or 1 or 0.5 or about 0.1% or less of the original spore.

Several different chemical methods, using a variety of chemical reagents, have been described. These methods attempt to produce empty exine shells from spores, for example:
F. Zetsche, P. Kalt, J. Liechti and E. Ziegler, J. Prakt. Chem., 148, (1937), 267-286, Saad M. Alshehri, Hamad A. Al-Lohedana, EidaAl-Farraj, Norah Alhokbany, Anis Ahmad Chaudhary, Tansir Ahamad. International Journal of Pharmaceutics 504, 39-47 (2016), Shashwati Atwe, Harvinder S. Gill, Yunzhe Ma, Patent WO2014062566 (A1), describes the use of sequentially, hot acetone, alkali and 85% phosphoric acid over several days; E. Dominguez, J. A. Mercado, M. A. Quesada and A. Heredia, Grana, Supplement 1, (1993), 12-17.) describes treating spores with anhydrous H F in pyridine; N. M. Tarlyn, V. R. Franceschi, J. D. Everard and F. A. Loewus, Plant, science, 90, (1993), 219-224, K. E. Espelie, F. A. Loewus, R. J. Pugmire, W. R. Woolfenden, B. G. Baldi and P. H. Given, Phytochemistry, 28, (1989), 751-753, and F. A. Loewus, B. G. Baldi, V. R. Franceschi, L. D. Meinert and J. J. McCollum, Plant Physiol., 78, (1985), 652-654.) describe treating spores with 4-methylmorpholine-N-oxide monohydrate; G. Erdtman, Svensk Botanisk Tidskrift, 54, (1960), 561-564, describes using a 9:1 mixture of acetic anhydride and concentrated sulfuric acid on spores, which have had contaminants mechanically removed; M. Couderchet, J. Schmalfus and P. Boger, Pesticide Biochemistry and Physiology, 55, (1996), 189-199, S. Gubatz, M. Rittscher, A. Meuter, A. Nagler and R. Wiermann, Grana, Supplement 1, (1993), 12-17, K. Schulze Osthoff and R. Wiermann, J. Plant Physiol., 131, (1987), 5-15 and F. Ahlers, J. Lambert and R. Wiermann, Z. Naturforsch., 54c, (1999), 492-495) describe methods which utilise enzymes; Michael G. Potroz, Raghavendra C. Mundargi, Jurriaan J. Gillissen, Ee-Lin Tan, Sigalit Meker, Jae, H. Park Haram Jung, Soohyun Park, Daeho Cho, Sa-Ik Bang Nam-Joon Cho Advanced Functional Materials 27 (2017), Raghavendra C. Mundargi, Michael G. Potroz, Jae Hyeon Park, Jeongeun Seo, Ee-Lin Tan, Jae Ho Lee & Nam-Joon Cho Sci Rep. 2016; 6: 19960, Arun Kumar Prabhakar, Hui Ying Lai, Michael G. Potroz, Michael K.Corliss, Jae HyeonPark, Raghavendra C. Mundargi, Daeho Cho, Sa-Ik, Bang, Nam-Joon Cho Journal of Industrial and Engineering Chemistry 53, 375-385 (2017) describes acidolysis in phosphoric acid at 70° C. over the range for 5-30 h. Gill, Harvinder Singh, Atwe, Shashwati U., Gonzalez-cruz, Pedro E. US 2018/0092852 A1 2018, Gonzalez-Cruz, P., Uddin, M. J., Atwe, S. U., Abidi, N. & Gill, H. S. ACS Biomaterials Science & Engineering 4, 2319-2329 (2018). describes using hot phosphoric acid before hot potassium hydroxide solution in contrast to earlier methods of base hydrolysis followed by phosphoric acid; Mujtaba, M., Sargin, I., Akyuz, L., Ceter, T. & Kaya, M. Materials Science & Engineering C-Materials for Biological Applications 77, 263-270 (2017), Mundargi, R. C. Raghavendra C. Mundargi, Michael G. Potroz, Jae Hyeon Park, Jeongeun Seo, Jae Ho Leeab and Nam-Joon Cho RSC Advances 6, 16533-16539 (2016), Park, J. H., Seo, J., Jackman, J. A. & Cho, N. J.. Scientific Reports 6 (2016) uses hot acetone followed by treatments with 4 M or 6 M HCl) at 70° C. for 10-48 h and a further 24 h in the acid.

The exine-construct of the present invention may be formulated with conventional additives appropriate for the application envisaged. In particular, there may advantageously be employed brightening agents such as optical brightening agents, fluorescent brightening agents and fluorescent whitening agents typically used to enhance the appearance of fabric and paper. Such agents are intended to cause a “whitening” effect by making materials look less yellow and increasing the amount of light reflected to the eye. Suitable brightening agents will be well known to those skilled in the art. Examples include stilbenes and fluorescent dyes such as umbelliferone, which adsorb energy in the UV portion of the spectrum and re-emit it in the blue portion of the visible spectrum. More specifically, there may be employed triazine-stilbenes (di-, tetra- or hexa-sulphonated), biphenyl-stilbenes, biphneylcoumarins, imidazolines, diazoles, triazoles and benzoxazolines.

The scope of the present invention is not limited by any particular usage of the exine shells construct. Examples of suitable uses are disclosed in WO-2005/000280, WO-2007/012856, WO-2007/012857 and PCT Application No. PCT/GB2008/004150, the contents of which are incorporated herein by reference.

In particular, the exine-construct may be used as a protection and/or delivery and/or removal vehicle.

There are inherent advantages to the use of naturally occurring exine shells as delivery vehicles, as described in WO-2005/000280 and WO-2007/012857. Because of its inherent non-toxicity, for instance, a spore-derived exine shell can be particularly suitable for use as a delivery vehicle in the context of formulations, which are likely to come into contact with, or be ingested by, the human or animal body. The proteinaceous materials, which can otherwise cause allergic reactions to spores, are removed preferably during the processes used to isolate the exine component.

Naturally occurring exine shells make ideal candidates for the systemic delivery of active substances such as pharmaceuticals or nutraceuticals.

The exine shells prepared from any given organism also tend to be closely uniform in size, shape and surface properties, unlike typical synthetic encapsulating entities. However, there is significant variation in spore size and shape, and in the nature of the pores in the exine shells, between different species, allowing an exine-construct according to the invention to be tailored dependent on the nature and desired concentration of the active substance, the site and manner of intended application, the desired active substance release rate, the likely storage conditions prior to use and the like.

It can also be possible to encapsulate relatively high quantities of an active substance within even a small exine shell. The combination of high active loadings, small exine shell size and adequate protective encapsulation is something that can be difficult to achieve using other known encapsulation techniques, and yet can be extremely useful in the context of preparing for example, pharmaceutical, veterinary or dietetic preparations, foods or beverages.

As described above, an exine shell is generally inert and non-toxic. Sporopollenin, for example, which is a component of most spore exine shells, is one of the most resistant naturally occurring organic materials known to man. It can survive very harsh conditions of pressure, temperature and pH as well as being insoluble in most inorganic and organic solvents (see G. Shaw, “The Chemistry of Sporopollenin” in Sporopollenin, J. Brooks, M. Muir, P. Van Gijzel and G. Shaw (Eds), Academic Press, London and New York, 1971, 305-348).

The ready, and often inexpensive, availability of spores, together with their natural renewable origin, also make them highly suitable for use as materials from which to produce the exine-constructs.

The exine-construct of the present invention may be particularly suitable as a delivery vehicle for a cosmetic substance, for which colour and/or fragrance is often of considerable importance. A cosmetic substance may for example be selected from makeup products (for example foundations, powders, blushers, eye shadows, eye and lip liners, lipsticks, other skin colourings and skin paints), skin care products (for example cleansers, moisturisers, emollients, skin tonics and fresheners, exfoliating agents and rough skin removers), fragrances, perfume products, essential oils, sunscreens and other UV protective agents, self-tanning agents, after-sun agents, anti-ageing agents and anti-wrinkle agents, skin lightening agents, topical insect repellents, hair removing agents, hair restoring agents and nail care products such as nail polishes or polish removers. A perfume product may comprise more than one fragrance.

The exine-construct of the invention may be used as a delivery vehicle in a toiletry product which may be selected from soaps; detergents and other surfactants; deodorants and anti-perspirants; lubricants; fragrances; perfume products; dusting powders and talcum powders; hair care products such as shampoos, conditioners and hair dyes; and oral and dental care products such as toothpastes, mouthwashes, breath fresheners and coatings for dental flosses and tapes.

The exine-construct of the invention may be used as a delivery vehicle in a laundry product, which may be selected from soaps; detergents and other surfactants; and conditioning agents such as fabric conditioners and softeners.

The exine-construct of the invention may be used as a delivery vehicle in a household product. Such a product may, for example, be selected from disinfectants and other antimicrobial agents, fragrances, perfume products, air fresheners, insect and other pest repellents, pesticides, laundry products (e.g., washing and conditioning agents), fabric treatment agents (including dyes), cleaning agents, UV protective agents, paints and varnishes.

The exine-construct of the invention may be used as a delivery vehicle in an agrochemical product. Such a product may, for example, be selected from fertilisers, plant growth regulators, insect and other pest repellents and pesticides including herbicides, fungicides, nematicides, rodenticides and insecticides.

The exine-construct of the invention may be used as a protection and/or delivery vehicle for an active substance that is physically or chemically attached to the exine shells and/or the exine-construct.

The active substance includes but is not limited to an agrochemical, pharmaceutical or veterinary (which includes drugs, vaccines and antibodies), cells, virus, genetic material, nutraceutical substance, a foodstuff, a household product ingredient, a laundry product ingredient, a construction material, a herbicide, a pesticide, a cosmetic product ingredient, a toiletry product ingredient whether living, non-living, monomeric, oligomeric or polymeric and whether organic, inorganic or organometallic and the exine-construct structure may for example take the form of a flat sheet, disc, rod or plate, tablet, capsule, lozenge or open bowl (well), box, sphere, brick, sinter, fits and filter and may have one or more hollow chambers and may be suitable and/or adapted and/or intended for anal, vaginal, oral, topical, intravenous, pulmonary, nasal, transdermal, subcutaneous, buccal, intramuscular, intraperitoneal or any other suitable form of delivery.

The active substance may itself be a naturally occurring substance or derived from a natural source, in particular a plant source.

A pharmaceutically or nutraceutically active substance may be suitable and/or intended and/or adapted for either therapeutic (e.g., healing, curing, remedial, medicinal, restorative, health-giving, tonic, sanative, reparative, corrective, ameliorative drugs) or prophylactic use such as a vaccine or antibody.

The exine-construct of the invention may be used as a protection and/or support and/or delivery vehicle for an active substance, such as a living cell, a dead cell, virus or bacteria; and/or the products or secretions of any of the aforementioned; an alcohol, antibiotic, drug, hormone or vaccine producing cell where the exine-construct will support encapsulated cell viability and function and protect encapsulated cells from the exine-construct external environment or from the human or animal immune system.

The exine-construct of the invention may be used as a delivery vehicle for an active substance that is a diagnostic agent, in particular one intended for oral ingestion. For instance, the active substance may be a radioactive tracer, or a magnetic tracer for use in magnetic resonance imaging. In such cases, a protective additive may help to ensure that the encapsulated active substance reaches its intended delivery site.

Particular examples of active substances include peptides (e.g., hormones such as insulin and growth hormones such as somatropin); antibodies; antibiotics, stem cells, cells, vaccines; enzymes (e.g., lactase and alkaline phosphatase); probiotics (e.g. Lactococcus lactis, a Gram-positive bacterium); and prebiotics (e.g. carbohydrates such as lactulose, lactitol oligofructose, inulin and galacto-oligosaccharides, tagatose, isomalto-oligosaccharides, polydextrose and maltodextrin).

The exine-construct of the invention may be used as a delivery vehicle for an active substance, which is a volatile substance, in particular a flavouring or fragrance.

An active substance may be chemically or physically bound to, an exine-construct according to the invention. Chemical and physical attachment may be achieved for example in the ways described in WO-2005/000280, WO-2007/012856 and WO-2007/012857; they may involve chemical modification of the exine shells comprising the exine-construct or at least their outer surface.

A conveniently, prepared exine-construct may be immersed in a solution or suspension of the relevant substance, which is then allowed to impregnate the exine-construct's chamber, suitably followed by a drying step to remove at least some of the residual solvent(s). Where the substance to be encapsulated is a liquid, such as an oil, the prepared exine-construct may simply be immersed in the liquid, which it will then absorb.

The exine-construct is suitably immersed in an excess of the substance to be encapsulated within it. One or more penetration enhancing agents may be used, again as described in WO-2005/000280, to aid impregnation into the exine-construct's chamber by the relevant substance. A reduced or increased pressure (with respect to atmospheric pressure) may instead or in addition be used to facilitate impregnation; for example, a mixture of an exine-construct and an active substance may be placed under vacuum in order to increase the rate of absorption of the active by the exine-construct.

A substance may be generated in situ within an exine-construct, for instance from a suitable precursor substance already associated with the exine shell. For example, a precursor substance may be chemically or physically bound to, or encapsulated within, an exine shell of the exine-construct, which is then contacted with a reactant substance that reacts with the precursor to generate the desired active substance or additive.

The exine-construct may be loaded with, or otherwise associated with, any suitable quantity of an active substance, depending on its intended use.

The exine-construct may be coated with a barrier layer to aid retention of the active substance, for example for further protection of an associated active substance, or to prevent its release until a desired time or location is reached. Such coatings may be as described in WO-2005/000280, WO-2007/012856 or WO-2007/012857

The hollow chambers of an exine-construct may be loaded with an active substance and the exine-construct then capped.

Where the exine-construct of the invention or the exine-construct structure is used as an antioxidant, it can simply be contacted with a substance or composition to be protected. Instead, or in addition, the substance or composition can be chemically or physically bound to, or loaded within, the exine-construct.

According to a further aspect of the invention there is provided a formulation comprising an exine-construct as herein described in association with an active sub stance.

A formulation according to this aspect of the invention may contain one or more additional agents for instance selected from fluid vehicles, excipients, diluents, carriers, stabilisers and surfactants.

A formulation according to this aspect of the invention may be an agrochemical product, a beverage product, a cosmetic product, a food product, a dietetic (which includes nutraceutical) product, toiletry products (e.g., a bath, soap, detergent, hair care or personal care product), laundry products (e.g. a soap; detergent, surfactants; fabric conditioner or softener) or a pharmaceutical or vaccine or veterinary product.

In another embodiment of the invention, the active substance comprises a living or a dead cell or virus or bacteria; and/or the products or secretions of any of the aforementioned. The active substance is a living or dead human cell, animal cell, algal cell, bacterial cell, fungal cell, protozoan cell or plant cell.

In another embodiment of the invention, the active substance comprises a living cell that is a hormone producing cell or a living cell that can differentiate into a hormone producing cell or the hormone produced by the living cell or a combination thereof.

In another embodiment of the invention, the active substance comprises a construction product.

In another embodiment of the invention, the active substance comprises a plant protection product.

In another embodiment of the invention, the active substance comprises a plant fertiliser product.

In another embodiment of the invention, the active substance comprises a plant growth regulator product.

In another embodiment of the invention, the active substance comprises a virus or viral material.

In another embodiment of the invention, the active substance comprises a vaccine material.

In another embodiment of the invention, the active substance comprises a contraceptive material.

In another embodiment of the invention, the active substance comprises genetic material.

For oral delivery, the formulation of the exine-construct may for example take the form of a capsule, disc or lozenge. Other suitable pharmaceutical and dietetic dosage forms are those disclosed in WO-2005/000280.

In accordance with the invention, exine-construct structures can be made by tightly compressing (or leaving under gravity) an amount of exine shells, which may have been coloured with such as a food dye, with other ingredients followed by treatment with such as a glue that contains a chemical bonding and/or physical binding agent that forms an attachment with the exine shells. The formation of an exine-construct structure may be by any method that is known in the art for forming such structures. A glue may be added before or after structure formation to allow for a chemical bond and/or physical attachment with the exine shells. The most common methods are by leaving under gravity or compression in a die mould. In either method, the components of an exine-construct structure are mixed, either wet or dry, and then (can be followed by drying in the case of wet mixing) an amount of the composition is applied to a mould under gravity or compression. A glue can be added at this stage and fixed at a later stage to form a chemical bond and/or physical attachment with the exine shells. Application of a glue can be added before or after the structure has been formed or the structure can be irradiated with such as UV light or heated to fix the glue. An exine-construct structure can also be coated as described above by those methods known in the art for coating similar structures. The most common method of making an exine-construct structure involves mixing the ingredients of the structure and compressing the mixture in a mould to give it the desired shape and hardness. The mixture of ingredients is usually mechanically compressed by a machine. The compressed mixture may be either wet or dry. However, in a method where the mixture is wet, the mixture or structure must be dried. Lubricants can be added to the composition that is to be formed into a structure to help reduce the frictional wear of the die and its associated parts. One or more glues can be added at any stage to make chemical and/or physical attachments with the exine shells.

One or more hollow inner chambers may be incorporated in the exine-construct structure by methods such as machining (including by methods such as abrasion, drilling, lathing or punching) of such as a rod, plate, sheet, tablet, capsule or lozenge exine-construct or by leaving a glue and exine shell mixture under gravity in a die mould or by compressing a glue and exine mixture in a die mould.

An exine-construct structure may also, but need not, include other ingredients. For example, the structure may include inactive fillers, chemical bonding and/or physical binding agents, e.g., glues, diluents, dispersants, lubricants, fats, waxes, polysaccharides, proteins, peptides, disintegrants, colouring agents and flavouring agents.

In accordance with the invention, exine shells may suitably be bound together chemically (involving ionic or covalent bonds) and/or physically (hydrogen bonding, van der Waals and hydrophobic-hydrophilic′ interactions) to provide an exine-construct, which may then be coloured with such as a food dye. The chemical and/or physical bonding can be achieved by: (i) leaving under gravity or compression in a mould with no addition of adhering composition, following by a bonding agent (e.g. glue); (ii) addition of an adhering composition (e.g., glue) to exine shells in the form of a powder or as a compressed disc; (iii) addition of an adhering composition (e.g.′ glue) to exine shells followed by leaving under gravity or compression in a mould. In any of the forgoing formulations, the adhering composition (e.g., glue) can be fixed by a reagent, catalyst or such at UV light or heat following formation of the exine-construct.

Other methods of production of exine-constructs are those as used in the plastics industry including 3D printing. The exine shells are well placed, as powders for use in known 3D printer technologies designed for powders. For example, “Powder-& Binder-based 3D Printing” is particularly applicable since in this technology a binder is used to “glue” parts together. A roller spreads a thin layer of powder on a platform and then a print head places a binding agent at specific points, printing a thin layer of a required form that is able to bind to subsequent layers. This process is then repeated until the required 3D shape is complete. In some cases, some post-processing such as post fusion with a glue might be required to provide additional strength. [https://imaterialise.helpjuice.com/design-printing/powder-based-3d-printing and https://ytec3d.com/oasis-3dp/retrieved 10/2/2020]

The exine-construct structure may for example take the form of a flat sheet, disc, rod or plate, tablet, capsule, lozenge, or open bowl (well) and may have one or more hollow inner chambers, box sphere, brick, sinter, frits and filter. The active substance may be attached chemically to the external surface of the exine-construct or encapsulated within its chamber, or chemically or physically bound to the inner chamber surface. The active substance may be loaded and/or encapsulated within the well or one or more inner chambers of an exine-construct structure; or the active substance is attached physically or chemically to a surface of the well or one or more inner chambers of the exine-construct structure.

Thus, the exine-construct of the invention offers an opportunity for a novel formulation capable of the encapsulation and/or delivery of active substances, which includes but is not limited to pharmaceutically active substances (which includes drugs, cells, vaccines and antibodies), nutraceutically active substances, dietetic active substances (which includes nutraceutically active substances), probiotics, cells (which includes human, animal, algal, bacterial, fungal, protozoan or plant cells), viruses, foods and food ingredients, food supplements, herbicides, pesticides and pest control agents, plant treatment agents such as growth regulators, antimicrobials, cosmetics (including fragrances), toiletries, disinfectants, detergents and other cleaning agents, construction materials, adhesives, diagnostic agents, dyes and inks, fuels, explosives, propellants and photographic materials. In general, the present invention may be used to protect any active substance encapsulated within an exine-construct, whether living, non-living, monomeric, oligomeric or polymeric and whether organic, inorganic or organometallic.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including any accompanying claims and drawings). Thus, features, integers, characteristics, compounds, chemical moieties or groups described in conjunction with a particular aspect, embodiment or example of the invention are to be understood to be applicable to any other aspect, embodiment or example described herein unless incompatible therewith. In addition the invention extends to any of the following in construction of the exine-constructs to include but not be limited to (i) variation of exine-construct assembly to include ionic forms of the exine shells used (e.g. salt or protonic forms); (ii) viscosity, concentration and type of chemical and/or physical binding agent used; (iii) temperature; (iv) pressure; (v) sequence or ratio of additions made in making formulations (e.g. mixtures of exine shell types, solvents or binding agents such as glues) of the exine-constructs.

Moreover, unless stated otherwise, any feature disclosed herein may be replaced by an alternative feature serving the same or a similar purpose.

### BRIEF DESCRIPTION OF THE FIGURES

The present invention will now be described by means of the following non-limiting examples, with reference to the following Figures in which:

FIG. 1 Shows an exine-construct as a well (or bowl) with a cap to form an enclosed chamber fabricated from exine shells and BlueSpot® superglue as a binder for the encapsulation of an aqueous dye.

FIG. 2a Shows an SEM image of the surface of an exine-construct disc fabricated from compressed exine shells and BlueSpot® superglue diluted 25% w/w in chloroform.

FIG. 2b Shows an SEM image of the surface of a disc as in 2 a, disc cut in half using a knife.

FIG. 3 Rate of absorption (over 70 min) at room temperature of PBS (pH 7.4) by exine-construct discs (100 mg in dry form) fabricated from compressed exine shells and BlueSpot® superglue diluted in 10% and 25% w/w respectively in chloroform.

FIG. 4 UV-Vis spectra of the passage of a solution of a peptide (enfuvirtide) through an exine-construct disc. The solid line shows the spectrum and absorption of the peptide solution before passage through the disc and the dotted line shows these features after passage. The spectrum and level of absorbance is much the same before and after passage indicating efficient migration at 0.5 mL/min under gravity with no loss or change in the peptide's spectrum.

The invention is further described by means of examples but not in any limitative sense.

### Example 1

Preparation of an Exine-Construct as a Water Permeable Well (or Bowl) with an Enclosed Chamber Using Exine Shells and Super Glue

Raw Lycopodium clavatum spores (500 g) were suspended in an aqueous solution of 9M-HCl (2.25 L) (1.687 mL 12 M HCl, 563 mL deionised water) stirred at 94° C. for 1 h. The spores were recovered by vacuum filtration (porosity grade 2) and washed with hot water (500 ml×2), (NB check the pH of filtrate was neutral), methanol (1L×2), acetone (1L×2). The final hydrolysed spore shells were dried under vacuum overnight (over phosphorous pentoxide) before further drying in an oven at 50° C. until constant weight. 200 g yield was recovered giving a 60% mass loss. The acid hydrolysed spore shells (10 g) were then stirred in 1 M NaOH (100 mL) and stirred for 5 min. The spore shells were recovered by vacuum filtration (porosity grade 2) and washed with hot water (500 ml×2), (NB check the pH of filtrate was neutral), methanol (1L×2), acetone (1L×2). The final hydrolysed exine shells were air dried under vacuum overnight (over phosphorous pentoxide) before further drying in an oven at 50° C. until constant weight. The density of the exine shells as a loose powder was 0.152 0.013) g.cm⁻³.

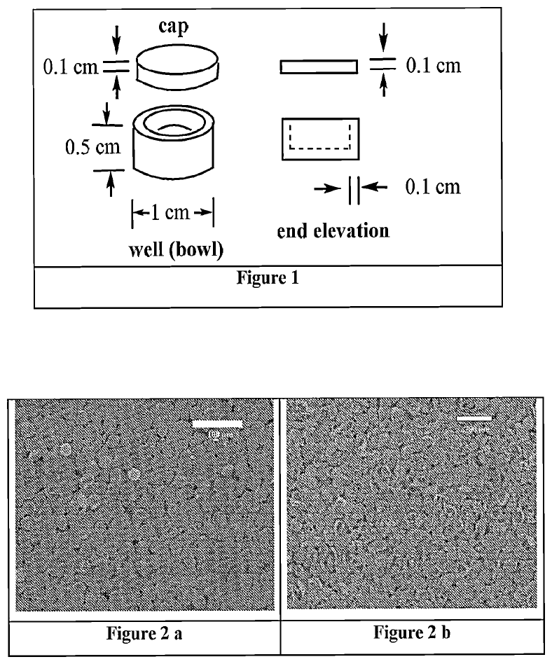

The exine shells were formed into two discs of 300 mg and 50 mg, respectively under a compression 6 tonnes cm⁻². The 300 mg disc was placed into 25% w/w of Blue spot® superglue in chloroform (2 mL) for 3 min and allowed to dry overnight. Once dry the disc was hollowed into the form of a well (FIG. 1). The 50 mg disc was placed into 10% w/w of Blue spot® superglue in chloroform (2 mL) for 3 min and allowed to dry overnight. The resulting disc formed a cap of the same diameter of the well (FIG. 1).

A blue water-soluble food dye (‘Essential Waitrose natural blue food colour’) was added into the disc well until it was nearly full. The well was sealed using Blue spot® superglue. After the glue had dried the sealed exine-construct containing the dye solution, was examined carefully to ensure the absence of leaks. The exine-construct was immersed in deionised water. After 1 h, the blue colour of the dye became evident in the water surrounding the dye filled exine-construct; the blue dye could be seen to diffuse uniformly from all outer surfaces of the exine-construct into the surrounding solution. After 12 h, the exine-construct was removed from the water and the cap of the exine-construct was cut open carefully with a scalpel. The colour of the solution inside the exine-construct and surrounding solution appeared to be of the same depth of blue colour, thus indicating diffusion across the exine-construct walls.

### Example 2

Sequestration of Nicotine into an Exine-Construct Disc Made from Whitened Exine Shells Compressed with Blue Spot® Superglue

Raw Lycopodium clavatum spores (199.3 g) were suspended in acetone (900 ml) and stirred at 60° C. for 4 hours. The spores were recovered by filtration (porosity grade 3) and washed with acetone (250 ml) and then left to dry. The spores were suspended in 6% KOH (w/v) (54.0 g, 900 ml) and stirred at 80° C. for 6 hours. The spores were recovered by filtration (porosity grade 3) and washed with hot water (500 ml×2). The spores were re-suspended in fresh 6% KOH and heated at 80° C. for a further 6 hours. The spores were recovered by filtration (porosity grade 3) and washed with hot water (500 ml×8), PBS (250 ml×2) and hot water (500 ml×2) and then suspended in EtOH (900 ml) and stirred at 80° C. for 4 hours. The spores were recovered by filtration (porosity grade 3) and washed ethanol (500 ml) and acetone (500 ml) and then dried under vacuum before further drying in an oven at 60° C. The resultant product was suspended in 50 ml of a 7% sodium hypochlorite solution and stirred at 60° C. for 2 hours. The bleached exine shells were recovered by filtration (porosity grade 3) and washed with water (50 ml×3), ethanol (50 ml×3) and acetone (50 ml×3), then dried overnight in open air and further dried under vacuum over P 2 0 5.

The bleached exine shells (79.7±1 mg) were compressed to 1 tonne.cm⁻² (1 cm circular cross section) connected to a vacuum (20 mm Hg) for 10-20 seconds. Blue spot® superglue in chloroform (1:9 w/w) was allowed to absorb into the disc and allowed to dry overnight. The resulting exine-construct disc was then immersed in 2 ml of a solution of nicotine (1 mg/mL) in water (pH 5.5) with stirring. After stirring for 24 h at room temperature 1.7±0.1 μs of nicotine (i.e., 14% w/w of the original concentration) remained in solution as determined by HPLC. The whitened exine-construct sequestered 86% of the nicotine.

### Example 3

Preparation of Exine-Constructs as Discs Using Exine Shells and Diluted Blue Spot® Superglue in Chloroform as a Glue and Rate of Absorption of Discs Immersed in Phosphate Buffer (PBS)

Lycopodium clavatum exine shells (100 mg) as prepared in Example 1, were compressed to 1 and 6 tonnes.cm⁻² as indicated in Table 1, in a die (1 cm circular cross section). The resulting discs were agitated for 3 min in solutions of 10% w/w and 25 w/w of Blue spot® superglue in chloroform (2 mL) respectively and heated to 50° C. for 12 h as indicated in Table 1. (NB FIG. 2a shows the face of the disc as produced as described using 6 tonnes.cm⁻² and 25% w/w of Blue spot® superglue in chloroform; FIG. 2b shows the cleaved face of the disc having been dissected with a knife).

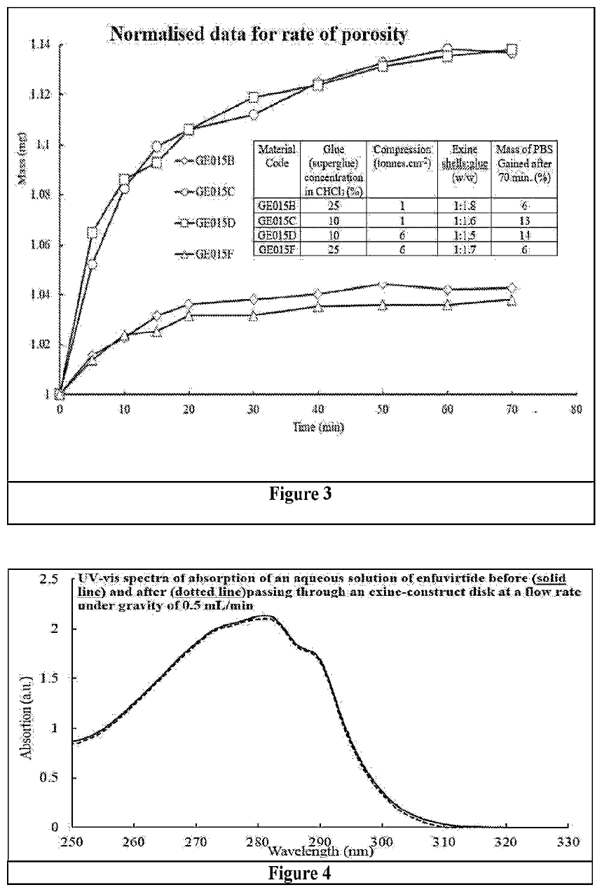

The discs were immersed in PBS (pH 7.4) gently swirled over 0-70 min as indicated in FIG. 3 to assess the rate of absorption of the PBS. Table 1 provides the total mass of PBS gained over agitation in PBS after 70 min. It was concluded that the concentration of superglue in the chlorinated solvent was a dominant feature of disc production under pressure in relation to the ability to absorb a surrounding aqueous solvent at room temperature.

TABLE 1

| | Glue (super glue) | Compression | Exine | Mass of PBS |
|---|---|---|---|---|
| Material | concentration in | (tonnes · | shells:Glue | gained after |
| code | CHCl₃ (%) | cm⁻²) | (w/w) | 70 min (%) |
| GE015B | 25 | 1 | 1:1.8 | 6 |
| GE015C | 10 | 1 | 1:1.6 | 13 |
| GE015D | 10 | 6 | 1:1.5 | 14 |
| GE015F | 25 | 6 | 1:1.7 | 6 |

### Example 4

Preparation of an Exine-Construct as a Well (or Bowl) with a Cap to Form an Enclosed Chamber Using Exine Shells and Superglue as a Glue for Encapsulation of

Brewer's Yeast as an Enclosed Fermentation Reactor Lycopodium clavatum exine shells (50 mg) as prepared in Example 1, were compressed to 6 tonnes.cm⁻² in a die (1 cm circular cross section). The resulting disc was agitated in a glue solution (25 w/w Blue spot® cyanoacrylate superglue in chloroform) (2 mL) for 3 min. to form a cap as in FIG. 1. A second disc was similarly formed from 300 mg exine shells and machined to form a well (or bowl) (FIG. 1) and filled with a dispersion of brewer's yeast (ca 1 mg/mL). The cap was sealed on to the top of the well with concentrated superglue. The sealed exine-construct was immersed in a ca 40% aqueous sucrose solution for 24 h. During this period, bubbles were produced over the whole surface of the exine-construct but were not assayed. The construct was removed from the sucrose solution and opened with a scalpel. The inside of the vessel was dry, indicating the formation of CO 2 displacing the aqueous content. In addition, the interior contained several large sucrose crystals on the inner walls of the exine-construct's cavity indicating the migration of sucrose solution across the exine-construct's walls.

### Example 5

Exine-Construct Disc Formation by a Sedimentation Methodology and Permeability of the Disc to an Aqueous Solution of a Protein (4565 g/Mol.)

Lycopodium clavatum exine shells (1.5 g) were prepared in accordance with Example 1 and measured into a 50 mL plastic centrifuge vial with a flat bottom. A glue solution was formulated by diluting Ametch® ethyl-cyanoacrylate (2.4 g) with chloroform to a total mass of 24 g. The glue solution was then added to the exine shells in the centrifuge vial and stirred gently to homogeneity. The vial was placed into an oven at 50° C. for a period of 16 h. The discs were then assessed for permeability of large molecules, using enfuvirtide (Fuzeon™) 4565 g/mol. Enfuvirtide (20 mg) was diluted into PBS 25 mL to give a 1.75×10⁻⁴ M solution, which was measured by UV-Vis absorption spectroscopy to give the solid line on FIG. 4. The enfuvirtide solution was placed into a tube, which contained a disc which was machined 1.3 cm in diameter and 1.2 mm thickness (density of 0.3 g/cm³) with a silicon washer above and below and then tightened into place with a screw cap with a hole in the middle. The rate of flow through the disc under gravity was measured to be approx. 0.5 mL per 5 minute and the solution, which passed through the disc was again measured by UV-vis to give the dotted line shown in FIG. 4.

1. An exine-construct comprising a plurality of exine shells bound together with an adhering composition.

2-3. (canceled)

4. An exine-construct according to claim 1 wherein the exine-construct is formed into a structure wherein said exine-construct structure comprises a well or one or more inner chambers.

5. An exine-construct according to claim 1 wherein an active substance is attached to an external surface of the exine shells or is encapsulated within the exine shells.

6-9. (canceled)

10. An exine-construct according to claim 1 wherein an active substance is attached to an external surface of the exine-construct.

11-13. (canceled)

14. An exine-construct according to claim 4 wherein an active substance is loaded and/or encapsulated within the well or one or more inner chambers and the exine-construct may be capped.

15. An exine-construct according to claim 4 wherein an active substance is attached to a surface of the well or one or more inner chambers of the exine-construct.

16-18. (canceled)

19. An exine-construct according to claim 1 wherein the exine-construct is in the form of a flat sheet, disc, rod or plate, tablet, capsule, lozenge, pessary, suppository or open bowl (well).

20. An exine-construct according to claim 1 wherein the exine shells are derivatised by hydrolysis, salt formation, protonation, deuteration, tritiation, esterification, amination, quarternisation, acetylation, sulfonation, sulfation, thiolation, alkylation, azidation, phosphorylation, nitration, metal chelation, halogenation, hydrogenation or chloromethylation or thiolation, or any combination thereof.

21. (canceled)

22. An exine-construct according to claim 1 wherein the active substance is either base or acid labile.

23. An exine-construct according to claim 1 wherein an external surface of the exine-construct is coated with a material to aid retention of the active substance.

24. An exine-construct according to claim 1 which is suitable and/or adapted and/or intended for anal, vaginal, oral, intravenous, pulmonary, nasal, topical, transdermal, buccal, subcutaneous, intramuscular, intraperitoneal or any other suitable form of delivery.

25. An exine-construct according to claim 1, for use in a method of surgery, therapy, prevention or diagnosis practised on a living plant, human or animal body.

26. A method for binding exine shells together involving attachment of the exine shells either directly or via bridging (or spacer) functional groups to provide an exine-construct wherein an adhering composition is used to attach the exine shells together to provide an exine-construct.

27. A method according to claim 26, wherein the exine shells are chemically and/or physically attached to provide an exine-construct.

28. A method according to claim 26, wherein the exine shells are left under gravity or compressed to provide an exine-construct.

29. A method according to claim 26, wherein the method comprises 3D printing.

30. (canceled)

31. A formulation comprising an exine-construct according to claim 1, in association with which is an active substance.

32. A formulation according to claim 31, which is an agrochemical product, a beverage product, a cosmetic product, a food product, a dietetic (which includes nutraceutical) product, toiletry products (e.g., a bath, soap, detergent, hair care or personal care product), laundry products (e.g. a soap; detergent, surfactants; fabric conditioner or softener) or a pharmaceutical or vaccine or veterinary product.

33. Use of an exine-construct according to claim 1 as an antioxidant.

34. Use of an exine-construct according to claim 1 as a protection and/or delivery vehicle for an active substance.

35. An exine-construct according to claim 1 for use in the manufacture of a medicament.

36. An exine-construct according to claim 14 for use in the manufacture of a medicament for the protection and/or delivery of a pharmaceutically active substance or a diagnostic agent to an animal or human patient.

37. An exine-construct according to claim 1, in the manufacture of a formulation for the protection and/or delivery of an active substance to a plant, human or animal.

38. An exine-construct according to claim 1 as a protection and/or delivery vehicle for a living or dead human, animal, algal, bacterial, fungal, protozoan, or plant cell; or virus.

39. An exine-construct according to claim 38 as a protection and/or support and/or delivery vehicle for an active substance, such as a living cell, a dead cell, virus or bacteria; and/or the products or secretions of any of the aforementioned; an alcohol, antibiotic, drug, hormone or vaccine producing cell.

40. An exine-construct according to claim 39 for use as a protection and/or support and/or delivery vehicle for an active substance, such as a living cell that is a hormone producing cell or a living cell that can differentiate into a hormone producing cell or the hormone produced by a hormone producing cell or a combination thereof.

41. An exine-construct according to claim 1, or a fragment thereof, in the removal of substances from solutions and emulsions.

42. An exine-construct according to claim 1, or a fragment thereof in the remediation of pollutants and contaminants.

43. An exine-construct according to claim 1, or a fragment thereof in the manufacture of components of machines and electrical/heat insulation units normally fabricated out of synthetic polymers.

44. A method for protecting an active substance from oxidation and/or for increasing the stability of the active substance or of a composition containing it, the method comprising adding an exine-construct to the substance and/or formulating the active substance with an exine-construct according to claim 1.

45. (canceled)

46. A method according to claim 44 wherein adding an exine-construct to the substance and/or formulating the active substance with the exine-construct increases the oxidative stability of a substance.

47. (canceled)
